(19) United States
(12) Patent Application Publication
(10) Pub. No.: US 2019/0008775 A1
(43) Pub. Date: Jan. 10, 2019

(54) Method for Preparing Modified Sodium Alginate Embolization Microsphere

(71) Applicant: Jiangnan University, Wuxi (CN)

(72) Inventors: Liping ZHANG, Wuxi (CN); Ren LIU, Wuxi (CN); Caihua NI, Wuxi (CN); Xue BAI, Wuxi (CN); Gang SHI, Wuxi (CN)

(21) Appl. No.: 15/756,021

(22) PCT Filed: Jan. 13, 2017
(86) PCT No.: PCT/CN2017/071080

§ 371 Date: Feb. 27, 2018

(30) Foreign Application Priority Data
Mar. 29, 2016 (CN) 201610188793.3

(51) Int. Cl. A61K 9/16; A61K 31/704; A61L 24/08; A61L 24/00

(52) U.S. Cl. CPC A61K 9/1652; A61K 9/1682; A61L 24/0042; A61L 24/08; A61K 31/704

(57) ABSTRACT

The present invention relates to a preparation method for modified sodium alginate embolization microspheres and a method for carrying an anti-cancer drug doxorubicin by means of modified sodium alginate embolization microspheres. The preparation method includes the following steps: (1) sodium alginate is modified by using taurine, so that modified sodium alginate is synthesized; (2) with high-concentration aqueous modified sodium alginate solution as a water phase, mineral oil as an oil phase and polyaldehyde cellulose as a crosslinking agent, modified sodium alginate embolization microspheres are prepared by an inverse emulsification method. By modifying sodium alginate by means of taurine, on one hand, sulfonic acid groups are introduced into the embolization microspheres, so that the drug-carrying rate is increased; on the other hand, the viscosity of a sodium alginate solution is decreased, which is favorable for the preparation of the high-concentration sodium alginate solution, and thereby regular sodium alginate embolization microspheres can be obtained.

### TECHNICAL FIELD

The present invention relates to a preparation method for a biodegradable drug carrier, which relates to the field of biological medicines, and in particular to a synthesis method for modified sodium alginate embolization microspheres.

### BACKGROUND

Hepatocellular carcinoma is one of malignant tumors which are common in stem cells. The cases of this tumor approximately accounts for 6% of diagnosed cancer cases in the world. Common tumor treatment protocols adopt surgical excision, but, for patients with cancers in middle and advanced stages, interventional therapies, such as transcatheter arterial chemoembolization (TACE) is an ideal treatment protocol. In this method, embolization microspheres which are injected into embolism of tumor tissues via a catheter not only block up nutrition from being supplied to the tumor tissues, but also can release an anti-tumor drug. As the concentration of the anti-cancer drug in the tumor tissues increases, the anti-cancer drug plays the role of inhibiting the diseased region, thus achieving the effect of treating the tumor.

Modified sodium alginate (SA), which is also called seaweed gel or alginic acid, is a natural biological macromolecular sodium salt extracted from natural brown seaweed. Because of its nontoxicity, good biocompatibility and wide source, modified sodium alginate has been widely applied in industries such as food, pharmacy, etc. Especially in the aspect of biomedical materials, modified sodium alginate has drawn much attention as a drug carrier. There have been some reports on non-drug-carrying embolization microspheres which are prepared with sodium alginate as a material. However, there are still defects in using pure sodium alginate to prepare drug-carrying embolization microspheres: (1) suitable drug-carrying groups are absent, the carboxyl group in sodium alginate is a weakly ionized group with weak interaction with a positive-charge drug, as a result, the carrying rate is limited, and the reaction rate is slow; (2) the viscosity of a pure sodium alginate solution is too high, emulsification time is too long, and therefore it is difficult to prepare embolization microspheres. Glutaraldehyde is a commonly used crosslinking agent, and is used to crosslink a high polymer, such as polyvinyl alcohol, chitosan, etc., but, because of its toxicity, glutaraldehyde has certain limitation in the field of medical material applications.

### SUMMARY

Aimed at the above-mentioned defects, this article first uses taurine (TA) to modify sodium alginate, so that modified sodium alginate (SA-TA) is obtained, and modified sodium alginate embolization microspheres are then prepared. The embolization microspheres can interact with an anti-tumor drug to serve as a drug carrier. The sodium alginate embolization microspheres have advantages such as nontoxicity, good biocompatibility, wide material sources, etc. After modification, modified sodium alginate molecules contain a large quantity of sulfonic acid groups. The sulfonic acid groups in taurine molecules are strongly ionized groups with extremely high hydrophilicity, and when being introduced into the sodium alginate molecules, the sulfonic acid groups can increase the drug-carrying rate of the modified sodium alginate embolization microspheres for the drug doxorubicin. Moreover, because of the existence of the sulfonic acid groups, the viscosity of modified sodium alginate in an aqueous solution is decreased to a certain degree, which makes possible the preparation of the sodium alginate embolization microspheres under the condition of high concentration. In crosslinking reaction, in order to avoid the toxicity of micromolecular glutaraldehyde, polyaldehyde cellulose is obtained by high polymer oxidation, and is added as a crosslinking agent into a modified sodium alginate solution, and the embolization microspheres are formed by a crosslinking-emulsion process.

The technical solution of the present invention is a preparation method for modified sodium alginate embolization microspheres, which includes the following steps in sequence:

(1) taurine and sodium alginate are subjected to amidation reaction, so that modified sodium alginate is obtained, adopted catalysts are 1-ethyl-3-(3-dimethylaminopropyl) carbodiiehydrochlide and N-hydroxysuccinimide, and the reaction is carried out in a phosphate buffer solution with a pH of 6.0;

(2) the modified sodium alginate product undergoes precipitation by using isopropanol, is re-dissolved by deionized water, is freeze dried after purification, and is re-dissolved, so that an aqueous modified sodium alginate solution is obtained.

(3) The high-concentration aqueous modified sodium alginate solution is dispersed into mineral oil for emulsification by adopting an inverse crosslinking-emulsion method, and polyaldehyde cellulose is then added as a crosslinking agent to prepare modified sodium alginate embolization microspheres.

As a preferred technical solution, in step (1), the weight ratio of sodium alginate to taurine is 5:1.49 to 5:5.96; the percentage concentration by weight of sodium alginate in the phosphate buffer solution is 1.8 wt %; the molar ratio of sodium alginate, 1-ethyl-3-(3-dimethylaminopropyl)carbodiiehydrochlide and N-hydroxysuccinimide is 1:1:1; and mechanical agitation is conducted under a temperature of 25° C. to carry out reaction for 24 h.

As a preferred technical solution, in step (2), the modified sodium alginate solution undergoes precipitation by using isopropanol which is three times the volume of the modified sodium alginate solution, and is then re-dissolved by using deionized water, so that a saturated solution is obtained, the operation is cycled for three times, and after 48 h of dialysis and freeze drying, the modified sodium alginate product is obtained.

As a preferred technical solution, in step (3), the high-concentration aqueous modified sodium alginate solution, the percentage by weight of which is 8 to 10 percent, is used to be dispersed into mineral oil, the volume proportion of oil and water is controlled at 5:1 to 10:1, Span 80, the percentage concentration by volume of which is 2%, is added as a stabilizer, and dispersion is conducted under the condition of 30° C. for 7 h.

As a preferred technical solution, the dosage of the added crosslinking agent polyaldehyde cellulose is 6% to 9% of the weight of modified sodium alginate, the crosslinking agent polyaldehyde cellulose is dissolved into a mixed solvent of deionized water and ethanol (the volume ratio is 1:1) in advance, and the solution is slowly dripped into the reaction system.

As a preferred technical solution, the preparation of polyaldehyde cellulose is carried out by the following steps:

(1) 2.08 g of sodium carboxymethylcellulose powder is added into a 250 ml flask, the viscosity of 20 g/L of used sodium carboxymethylcellulose in water is 300 mpa·s to 800 mpa·s, 80 mL of distilled water is then added, and stirring is constantly conducted under 25° C. until the sodium carboxymethylcellulose powder is completely dissolved;

(2) 1.5 g of sodium periodate is dissolved into 20 ml of distilled water, the solution is then slowly added into the flask, and the reaction continuously goes on under 25° C. for 24 hours;

(3) 20 mL of glycol is then added into the flask to stop the reaction; 30 minutes later, the mixture is poured into a dialysis bag (MWCO 3500) and thoroughly dialyzed in distilled water; and finally, by freeze drying, a product, i.e. polyaldehyde cellulose, is obtained.

Disclosed is a method for carrying an anticancer drug doxorubicin by means of modified sodium alginate embolization microspheres, which is characterized in that the drug-carrying method is an ion exchange method, doxorubicin (positively charged) and sulfonic acid groups (negatively charged groups) contained in the solution realize drug carrying by electrostatic adsorption, and the drug-carrying rate is up to 35 percent.

Specifically, in step (1), the reaction system of amidation reaction includes a phosphate buffer solution (PBS, pH 6.0), 1-ethyl-3-(3-dimethylaminopropyl)carbodiiehydrochlide (EDC.HCl) and N-hydroxysuccinimide (NHS), mechanical agitation is conducted under the condition of 25° C. for 24 h, wherein the molar ratio of fed sodium alginate constitutional unit, 1-ethyl-3-(3-dimethylaminopropyl) carbodiiehydrochlide and N-hydroxysuccinimide is 1:1:1.

Specifically, in step (2), after the amidated modified sodium alginate product undergoes isopropanol precipitation and redissolution for three times, dialysis is conducted in ultrapure water for 24 h.

Specifically, in step (3), the aqueous modified sodium alginate solution is adopted as a water phase, mineral oil (paroline) is adopted as an oil phase, and the proportion of oil and water is controlled at 5:1 to 10:1; Span 80, the percentage concentration by volume of which is 2%, is added as a stabilizer; after 7 h of dispersion under the condition of 30° C., polyaldehydecellulose is added as a crosslinking agent, and after 20 h of reaction, the modified sodium alginate embolization microspheres are obtained.

Modified sodium alginate has —COOH groups existing in a phosphate buffer solution (pH 6.0), —NH₂ groups are contained in taurine molecules, the amino groups and the carboxyl groups can generate amidation reaction under certain conditions, generating an amidated product, and when the molar weights of sodium alginate and taurine participating in the amidation reaction are different, amidated products at different degrees of reaction can be obtained. Therefore, when the present invention designs a synthesis formula, different molar ratios of fed sodium alginate and taurine are adopted, so that amidated products of modified sodium alginate with different sulfonic acid group contents can be effectively obtained.

The present invention further provides an application of the modified sodium alginate embolization microspheres in chemotherapeutic drug carriers. Being guided into blood vessels around tumor tissues, the modified sodium alginate embolization microspheres not only block up nutrition from being supplied to the tumor tissues, but also can release an anti-tumor drug, and as the concentration of the anti-cancer drug in the tumor tissues increases, cancer cells can be killed. The modified sodium alginate embolization microspheres can be completely degraded in the body and discharged out of the body by metabolism.

By means of the above-mentioned solution, the present invention at least has the following advantages:

1. As the surfaces of the embolization microspheres contain the carboxyl groups and the sulfonic acid groups, the interaction with the anti-tumor drug doxorubicin can be enhanced, and therefore the drug-carrying rate of the carrier can be increased.

2. As the surfaces of the embolization microspheres contain the sulfonic acid groups to adsorb drug molecules, drug leakage caused by the physical adsorption of the surfaces of the embolization microspheres is greatly eliminated.

3. By replacing glutaraldehyde with the crosslinking agent polyaldehyde cellulose, toxicity is prevented.

4. The modified sodium alginate embolization microspheres are nontoxic, has good cytocompatibility, and meets the safety standard for use in the human body.

5. When the amidated product is used to synthesize the embolization microspheres, the method is simple, the conditions are mild, any catalyst and other additives are not needed, no byproduct is produced, the reaction is sufficient, and the product is pure.

The description above is merely the summery of the technical solution of the present invention, and in order to more clearly understand the technical means of the present invention and implement the technical means according to the content of the description, detailed description is made below with preferred embodiments of the present invention in cooperation with drawings.

### BRIEF DESCRIPTION OF FIGURES

FIG. 1 is a synthesis route for modified sodium alginate.

FIG. 2 is an infrared spectrogram of sodium alginate before and after modification, wherein a: sodium alginate (SA); b: taurine (TA); and c: amidated product ST11 of sodium alginate and taurine.

FIG. 3 is a super-depth-of-field microscope picture of a modified sodium alginate embolization microsphere in the present invention, wherein a: before drug carrying; b: 10 min after drug carrying; c: 24 h after drug carrying; and d: embolization microsphere section.

FIG. 4 is a drug-carrying curve of the modified sodium alginate embolization microspheres in the present invention.

FIG. 5 is a cumulative release rate curve of the modified sodium alginate drug-carrying embolization microsphere ST11 in the present invention in release media with different pHs.

FIG. 6 is drug release curves of drug-carrying embolization microspheres produced by reactions between taurine and sodium alginate according to different molar ratios in in-vitro simulated body fluid, wherein ST10, ST11, ST12 and ST21 respectively represent the embolization microspheres prepared from amidated products when the weight ratios of fed sodium alginate and taurine in amidation reactions are 5:0, 5:1.49, 5:2.98 and 5:5.96.

FIG. 7 is a cytotoxicity result of the modified sodium alginate embolization microspheres in the present invention.

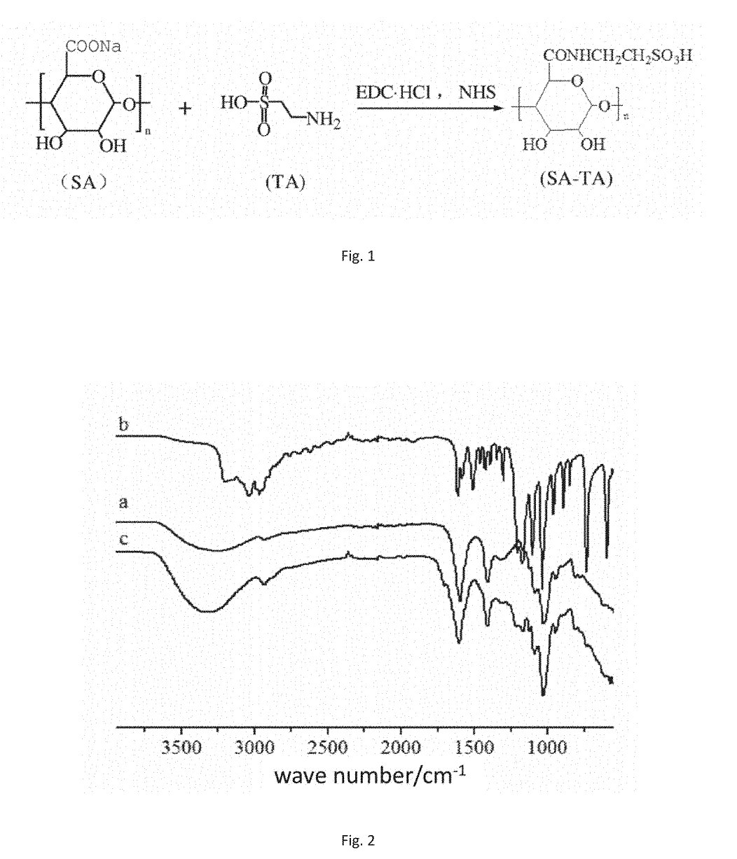

### DETAILED DESCRIPTION

The specific embodiments of the present invention are further described in details below in reference to the drawings and embodiments. The embodiments below are used to describe the present invention rather than limit the scope of the present invention.

### Example 1

### (1) Preparation of Modified Sodium Alginate:

5 g of sodium alginate (SA) is added into 300 mL of phosphate buffer solution (PBS, pH 6.0) and completely dissolved, 1-ethyl-3-(3-dimethylaminopropyl)carbodiiehydrochlide(EDC.HCl) is then added, N-hydroxysuccinimide (NHS) and taurine (TA) are added after 20 min of mechanical agitation, wherein the molar ratio of fed sodium alginate (SA), 1-ethyl-3-(3-dimethylaminopropyl)carbodiiehydrochlide (EDC.HCl) and N-hydroxysuccinimide (NHS) is 1:1:1, and reaction is carried out under 25° C. for 24 h. When the reaction is complete, isopropanol which is two to three times the volume of the reaction solution is used for precipitation, deionized water is then used for redissolution, so that a saturated solution is obtained, the operation is cycled for three times, and after 48 h of dialysis and freeze drying, a modified sodium alginate product is obtained. As shown in table 1, the amount of added taurine is regulated, so that a series of modified sodium alginate products are obtained.

TABLE 1
Modified Sodium Alginate Synthesis Formula and Element
Analysis Result

| Sample | SA/g | TA/g | S/% | N/% |
|---|---|---|---|---|
| ST10 | 5 | 0 | 0.523 | 0.06 |
| ST21 | 5 | 1.49 | 1.465 | 1.543 |
| ST11 | 5 | 2.98 | 2.192 | 1.806 |
| ST12 | 5 | 5.96 | 5.898 | 3.729 |

SA and TA respectively represent sodium alginate and taurine.

(2) Preparation of Polyaldehyde Cellulose:

2.0 g of sodium carboxymethylcellulose powder is weighed and added into a 250 mL flask, the viscosity of 20 g/L of sodium carboxymethylcellulose is 300 mpa·s to 800 mpa·s, 80 mL of distilled water is then added, and constant stirring is conducted under 25° C. until it is completely dissolved; 1.5 g of sodium periodate is dissolved into 20 ml of distilled water, the solution is then slowly added into the flask, and the reaction continuously goes on under 25° C. for 24 hours; 20 mL of glycol is then added into the flask to stop the reaction; 30 minutes later, the mixture is poured into a dialysis bag (MWCO 3500) and thoroughly dialyzed in distilled water; and finally, by freeze drying, a product, i.e. polyaldehyde cellulose, is obtained.

(3) Preparation of Modified Sodium Alginate Embolization Microspheres:

The above-mentioned modified sodium alginate ST10 is prepared into an aqueous solution, the concentration by weight of which is 8%, 5 mL of the solution is added into 50 mL of liquid paraffin containing 2% (v/v) of Span 80, and after uniform dispersion, 3 mL of polyethylene glycol is added. Emulsification is carried out under the condition of 30° C. for 4 h, the crosslinking agent polyaldehyde cellulose is added, its dosage is 6 to 9 percent of the weight of modified sodium alginate, the crosslinking agent polyaldehyde cellulose is dissolved into a mixed solvent of deionized water and ethanol (the volume ratio is 1:1) in advance, the solution is slowly dripped into the reaction system, crosslinking reaction is carried out for 24 h, n-hexane and isopropanol are sequentially used for washing for three times after the reaction is completed, and after filtration, vacuum drying is conducted.

### Example 2

The weight ratio of modified sodium alginate to taurine is 5:1.49, and the other synthesis process is the same as that in example 1.

### Example 3

The weight ratio of modified sodium alginate to taurine is 5:2.98, and the other synthesis process is the same as that in example 1.

### Example 4

The weight ratio of modified sodium alginate to taurine is 5:5.96, and the other synthesis process is the same as that in example 1.

### Example 5

A certain amount of modified product ST10 and a certain amount of modified product ST11 are respectively weighed and prepared into aqueous solutions, the parts by weight of which are 1%, 2%, 3%, 4%, 5%, 6% and 8%, and a viscometer is used to measure their viscosity changes separately.

TABLE 2
Viscosity Change of Aqueous Sodium Alginate
Solution Before and After Modification

| | Sample Concentration (wt %) | | | | | |
|---|---|---|---|---|---|---|
| | 2% | 3% | 4% | 5% | 6% | 8% |
| SA/mpa · s | 6.9 | 14.2 | 42.8 | 116 | 151 | 190 |
| ST11/mpa · s | 5.4 | 8.6 | 17.8 | 35.4 | 54 | 114 |

Table 2 is the change of the aqueous solution viscosity of sodium alginate before and after modification under the condition of 25° C. It can be observed from the table that the viscosity of modified sodium alginate is obviously decreased in comparison with the viscosity of unmodified sodium alginate, and this indicates that the viscosity of modified sodium alginate is decreased because of the existence of sulfonic acid groups. In the process of the experiment, as the viscosity of modified sodium alginate is decreased, the preparation of a high-concentration aqueous modified sodium alginate solution is made possible.

### Example 6

After SA, TA and ST11 are respectively purified and freeze-dried, a total-reflection Fourier infrared spectrometer is used to perform infrared scanning within a wave number range between 4000 cm⁻¹ and 500 cm⁻¹, so that an infrared spectrogram is obtained.

As shown in FIG. 2, there are obvious differences between the curve a, the curve b and the curve c, an amide l band characteristic absorption peak with weak intensity appears at 1685 cm⁻¹ in c, moreover, a characteristic absorption peak of the amide group will appear at 3500 cm⁻¹ to 3300 cm⁻¹, it can be seen from the comparison between a and c that the peak at this point of c is remarkably enhanced, and this indicates the existence of the amide group in c. By comparing b and c, the double peaks of the amino group at 3000 cm⁻¹ in c disappear, and this also indirectly proves the generation of the amide group. It can be known from this that the amide group is successfully introduced into SA-TA.

### Example 7

20 mg of dried and screened blank embolization microspheres are accurately weighed, added into 10 mL of doxorubicin hydrochloride solution with a concentration of 1.5 mg/mL and magnetically stirred in the dark under room temperature, the color of the doxorubicin hydrochloride solution gradually becomes light, and the color of the embolization microspheres is deep red. An ultraviolet/visible light spectrophotometer is used to detect the content of doxorubicin hydrochloride in the embolization microspheres at the wavelength of 483 nm, and a drug-carrying curve of the embolization microspheres is drawn.

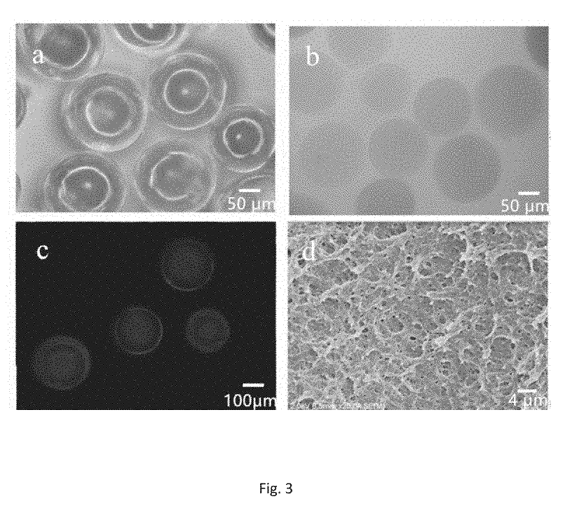

a, b and c in FIG. 3 respectively represent the morphologies of the embolization microspheres before drug carrying, 10 min after drug carrying and 24 h after drug carrying. It can be seen from the drawing that as drug-carrying time goes on, the color of the embolization microspheres is gradually deepened, this is mainly because the color of doxorubicin is red; in the drug-carrying process, as time goes on, the amount of the drug carried by the embolization microspheres increases, and the color of the embolization microspheres also deepens accordingly; FIG. d is the internal section of the embolization microsphere, and it can be seen from the drawing that a lot of tiny hollow structures exist in the embolization microsphere.

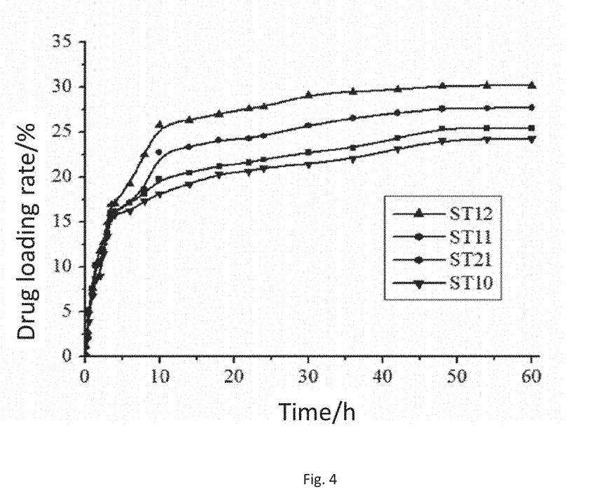

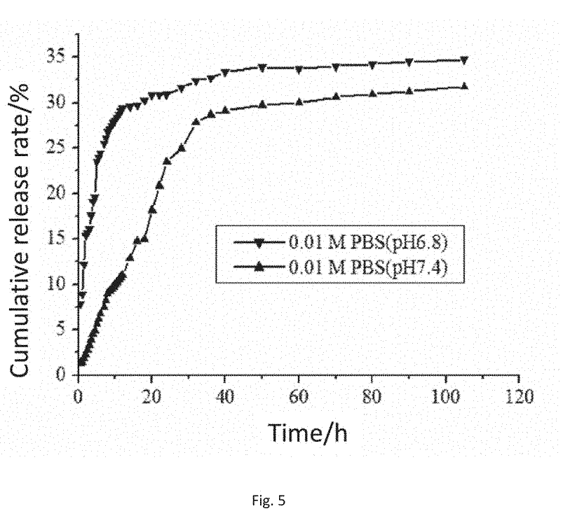

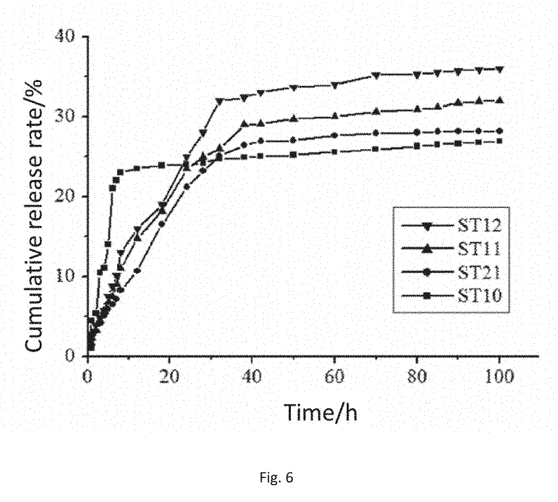

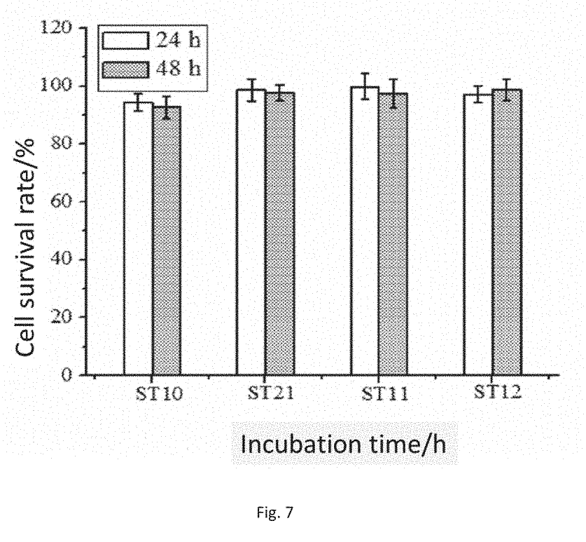

FIG. 4 is a diagram of the drug-carrying condition of the modified sodium alginate embolization microspheres, wherein the drug-carrying rate of ST10 is the lowest, and the drug-carrying rate of ST21 is the highest. In the carrying process, the drug-carrying speed is high at the beginning, and starts to tend to be steady and slow after about 10 h. This is because an ion exchange method is adopted when the embolization microspheres carry the drug, and the more the sulfonic acid group content in the embolization microspheres is, the higher the capability of ion exchange with doxorubicin will be and the higher the drug-carrying rate will be.

### Example 8

20 mg of drug-carrying embolization microspheres ST10, 20 mg of drug-carrying embolization microspheres ST21, 20 mg of drug-carrying embolization microspheres ST11 and 20 mg of drug-carrying embolization microspheres ST12 are respectively weighed and poured into 20 mL of 0.0M PBS (pH 7.4) solution, the solutions are put into a constant-temperature water bath oscillator, temperature is controlled at 37±0.5° C., 5 mL of supernate is weighed at each fixed point, drug contents in the buffer solutions are detected by an ultraviolet/visible light spectrophotometer, the operation is repeated for three times, mean values are obtained, and cumulative release amounts are calculated according to a formula below. Each time after sampling, fresh release solution of the same volume is added.

The drawing shows the drug release conditions of the different modified sodium alginate embolization microspheres in 0.01M PBS (pH7.4). In the drawing, the release of the embolization microspheres ST10 reaches maximum within initial 12 h, and then slowly tends to be gentle while the cumulative drug release amounts of the other proportions of embolization microspheres reach maximum after about 32 h, and this indicates that the functional modified embolization microspheres have a slow release effect. The main reason is that the sulfonic acid groups adsorb drug molecules by charge adsorption, consequently, sudden release caused by the physical adsorption of the surfaces of the embolization microspheres is greatly eliminated, and the embolization microspheres play the role of slowly releasing the drug.

### Example 9

Cytotoxicity of Modified Sodium Alginate Embolization Microspheres:

In a water bath kettle with a temperature of 37° C., 3T3 cells cryopreserved under −80° C. are rapidly thawed, transferred into a centrifuge tube containing 7 mL of RPMI-1640 culture solution and centrifuged at 800 rpm, an RPMI-1640 culture solution containing 10% of calf serum is used to blow the cells, so that a single-cell suspension is prepared, and the single-cell suspension is transferred into a 50 mL culture flask and cultured in an incubator with 5% of CO₂ under 37° C.

With the extract of the modified sodium alginate embolization microspheres in normal saline as a studied object, an MTT method is adopted to test the cytotoxicity of the produced extract, mouse fibroblasts are inoculated at about 1.2×10⁵/mL into a 96-well plate, 100 μL per well, and are respectively cultured for 24 h and 48 h, the raw culture solution in each well is sucked out, 100 μL of negative control solution (the sample set is the RPMI-1640 culture solution containing 10% of calf serum), positive control solution (0.64% of phenol medium) and sample sets (the sample sets are the RPMI-1640 culture solutions respectively containing 10% of calf serum (t=24 h and t=48 h)) are added into each well, culture is continued in the incubator with 5% of CO₂ under 37° C., and culture is respectively conducted for 24 h and 48 h. Four parallel wells are arranged in each group. After being taken out, the culture plate is observed under an inverted microscope, and the growth conditions of the cells are evaluated. 20 μL of MTT is then added. After culture is continued for 4 h, the liquid in the cells in the culture plate is completed sucked out, dimethyl sulfoxide is then added, a microplate reader is used to measure absorbance values (A) at 570 nm, and the survival rate of the cells is calculated. In terms of cell survival rates of 3T3 in different concentrations of embolization microsphere extracts, after the 3T3 cells cultured in the embolization microsphere extracts (24 h and 48 h) are incubated under 37° C. for 24 h, the relative growth rates of the cells all reach 90% or more, and this indicates that the growth states of the 3T3 cells in the cell culture media diluted by the extracts are good, indicating that the embolization microspheres do not have cytotoxicity and are good in cytocompatibility.

What is claimed is:

1. A method for preparing modified sodium alginate embolization microspheres, comprising the following steps:
(1) subjecting taurine and sodium alginate to an amidation reaction, and obtaining a modified sodium alginate product, and wherein adopted catalysts are 1-ethyl-3-(3-dimethylaminopropyl) carbodiiehydrochlide and N-hydroxysuccinimide, and a reaction is carried out in a phosphate buffer solution with a pH of 6.0;
(2) precipitating the modified sodium alginate product by using isopropanol, re-dissolving the modified sodium alginate product by deionized water, freeze drying the modified sodium alginate product after purification, and re-dissolving the modified sodium alginate product, and obtaining an aqueous solution of modified sodium alginate;
(3) dispersing high-concentration of the aqueous solution of modified sodium alginate into mineral oil for emulsification by adopting an inverse crosslinking-emulsion method, adding polyaldehyde cellulose as a crosslinking agent, and obtaining modified sodium alginate embolization microspheres.

2. The method according to claim 1, wherein in step (1), the weight ratio of sodium alginate to taurine is 5:1.49 to 5:5.96; the weight percentage concentration of sodium alginate in a phosphate buffer solution is 1.8 wt %; the molar ratio of sodium alginate, 1-ethyl-3-(3-dimethylaminopropyl) carbodiiehydrochlide and N-hydroxysuccinimide is 1:1:1; and the method further comprises conducting mechanical agitation under a temperature of 25° C. to carry out reaction for 24 hours.

3. The method according to claim 1, wherein in step (2), the method further comprises precipitating the aqueous solution of modified sodium alginate by using isopropanol which is three times the volume of the aqueous solution of modified sodium alginate, re-dissolving by using deionized water, obtaining a saturated solution, repeating the operation for three times, and after 48 hours of dialysis and freeze drying, obtaining the modified sodium alginate product.

4. The method according to claim 1, wherein in step (3), the method further comprises dispersing the high-concentration of the aqueous solution of the modified sodium alginate with a weight percentage of 8 to 10 percent into mineral oil, controlling the volume proportion of oil and water to be at 5:1 to 10:1, adding Span 80 with a volume percentage of 2% as a stabilizer, and conducting dispersion under the condition of 30° C. for 7 hours.

5. The method according to claim 1, wherein amount of added crosslinking agent polyaldehyde cellulose is 6% to 9% of the weight of the modified sodium alginate, and the method further comprises dissolving the crosslinking agent polyaldehyde cellulose into a mixed solvent of deionized water and ethanol with a volume ratio of 1:1 in advance, and slow-dripping a solution thereof into a reaction system.

6. The method according to claim 1, wherein preparing polyaldehyde cellulose is carried out by the following steps:
(1) adding 2.0 g of sodium carboxymethylcellulose powder into a flask of 250 ml, wherein a viscosity of 20 g/L of sodium carboxymethylcellulose thereof in water is 300 mpa·s to 800 mpa·s, adding 80 mL of distilled water, and stirring constantly under 25° C. until the sodium carboxymethylcellulose powder is completely dissolved;
(2) dissolving 1.5 g of sodium periodate into 20 ml of distilled water, adding a solution thereof slowly into the flask, and continuing the reaction under 25° C. for 24 hours;
(3) adding 20 mL of glycol into the flask to stop the reaction; 30 minutes later, pouring a mixture thereof into a dialysis bag (MWCO 3500) and thoroughly dialyzing in distilled water;
and finally obtaining a product of polyaldehyde cellulose by freeze drying.

7. A method for carrying an anticancer drug doxorubicin by the modified sodium alginate embolization microspheres prepared according to claim 1, comprises using an ion exchange method as a drug-carrying method, using positively-charged doxorubicin and negatively-charged sulfonic acid groups through electrostatic absorbing and carrying the anticancer drug contained in a solution, and realizing a drug-carrying rate of up to 35%.
